(19) United States
(12) Patent Application Publication
(10) Pub. No.: US 2009/0059163 A1
(43) Pub. Date: Mar. 5, 2009

(54) Ophthalmic Lens Having Selected Spherochromatic Control and Methods

(76) Inventor: Candido D. Pinto, Penfield, NY (US)

Correspondence Address: Bausch & Lomb Incorporated, One Bausch & Lomb Place, Rochester, NY, 14604-2701, US

(21) Appl. No.: 12/194,956

(22) Filed: Aug. 20, 2008

Related U.S. Application Data
(60) Provisional application No. 60/968,905, filed on Aug. 30, 2007.

(51) Int. Cl. G02C 7/02; A61F 2/16

(52) U.S. Cl. 351/159; 623/6.23

(57) ABSTRACT

An aspect of the invention is directed to an ophthalmic lens, comprising at least one optic having at least one aspheric surface, the lens configured such that, when applied to an average eye, for a bandwidth between a wavelength of 656 nm and a wavelength of 486 nm, at a location disposed in a range 0.7 to 1.0 of the normalized clear aperture of the lens, the spherochromatism being less than 25 waves of the center wavelength of the bandwidth. In some embodiments, the lens is multizonal.

### CROSS-REFERENCE

This application claims the benefit of Provisional Patent Application No. 60/968,905 filed Aug. 30, 2007 which is incorporated by reference herein.

### FIELD OF INVENTION

The present invention relates to ophthalmic lenses, and more particularly to ophthalmic lenses designed to address spherochromatism.

### BACKGROUND OF THE INVENTION

Aberrations are present in all optical systems. A lens designer is faced with using a limited number of degrees of design freedom (e.g., surface curvatures, thickness, indices of refraction) to balance or eliminate individual aberrations. For a given design application, the designer is required to make tradeoffs regarding the degree to which a given aberration is to be corrected and/or tolerated (i.e., the degree to which the aberration is controlled).

Different tradeoffs of aberrations have been found to be appropriate for different types of instruments. For example, telescopes and microscopes are designed to present an eye with images showing fine details for intent examination by an observer. Similarly, lithographic projectors project images with fine details for accurate production of integrated circuits. In the above instruments, the finely-detailed images are generated by lens systems having numerous optical elements which allow a designer many degrees of freedom.

Ophthalmic lens systems present unique design challenges. Such lens systems typically include only a single optic of limited thickness which provides more limited design freedom than the systems discussed above; furthermore the ophthalmic lens systems have limited optomechanical stability (i.e., the lenses are subject to considerable movement relative to the systems discussed above). Additionally, ophthalmic lenses are designed to provide for comfortable vision by a wearer under a variety of circumstances. For example, it may be desirable that a given ophthalmic lens provide comfortable vision during reading or distant vision, and in high or low light conditions. Given the above design limitations and the nature of human vision, ophthalmic lenses are generally designed for more cursory vision than other optical systems.

To date, the degrees of correction of individual aberrations in ophthalmic lenses that make for comfortable and high quality vision have been the subject of conjecture. A complication of determining corrections to be made in ophthalmic lenses is that suitability of aberration corrections is determined to a significant degree by a wearer's brain's interpretation of an image presented to the wearer's retina by the lens.

One example of the uncertainty of selecting suitable degrees of correction (i.e., selecting the degrees of control) relates to correction (i.e., control) of the spherical aberration. For example, some have speculated that ophthalmic lenses should be designed to compensate for an eye's inherent spherical aberration to minimize spherical aberration at the center of visible bandwidth of light (approx. 550 nm); and, others have speculated that a selected amount of overcorrection or undercorrection is appropriate for comfortable, quality vision.

### SUMMARY

Aspects of the present invention are directed to an optical aberration known as spherochromatism, also referred to as wavelength-dependent spherical aberration. For example, manifestations of spherochromatism are particularly acute while driving at night, when a driver's pupils may be enlarged. Under such conditions, bright lights (e.g., headlights of on-coming automobiles) form halos of different colors (e.g., the headlights appear as blue halos surrounding white or yellow disks). Spherochromatism has also been found to be particularly acute with multifocal lenses, where halos of different colors from each of the foci of the lens overlap thereby compounding the problems arising from spherochromatism.

Aspects of the present invention are directed to ophthalmic lenses having selected correction characteristics (i.e., control) of the aberration spherochromatism. Embodiments of lenses may be configured such that, when an ophthalmic lens is added to an average eye, the amount of spherochromatism of the eye (including the ophthalmic lens, and other eye components) is substantially zero. In other embodiments, lenses may be configured to impart on a wavefront an amount of spherochromatism that at least partially offsets spherochromatism present in a wearer's eye. The term “ophthalmic lens” includes but is not limited to intraocular lenses (IOLs), contact lenses, and corneal onlays or inlays.

An aspect of the invention is directed to an ophthalmic lens, comprising at least one optic having at least one aspheric surface, the lens corrected (i.e., the lens configured) such that, when the lens is placed applied to an average eye, for a bandwidth between a wavelength of 656 nm and a wavelength of 486 nm, at locations disposed in a range 0.7 to 1.0 of the normalized clear aperture of the lens, the spherochromatism is less than 25 waves of the center wavelength of the bandwidth.

In some embodiments, the spherochromatism is less than 10 waves. In some embodiments, the spherochromatism is less than 5 waves.

In some embodiments, the clear aperture of the lens is 6.0 mm.

The lens may be a contact lens, an IOL or other suitable ophthalmic lens.

In some embodiments, the lens has a non-zero amount of spherical aberration for at least one wavelength in the bandwidth.

In some embodiments, the lens is configured such that for a bandwidth between a wavelength of 700 nm and a wavelength of 400 nm, at locations disposed in a range 0.7 to 1.0 of the normalized clear aperture of the lens, the spherochromatism is less than 25 waves of the center wavelength of the bandwidth.

In some embodiments, the lens provides a polychromatic modulation that is greater than 10% for a test target having a modulation of 100 line-pairs/mm for light in the bandwidth. It will be understood that 100 line-pairs/mm corresponds to “20/20 vision” test pattern.

The sag of the aspheric surface may be described by the following equation.

z_{Schmidt}(r)=z_{s tan dard}(r)+α₁r²+α₂r⁴+α₃r⁶+α₄r⁸+α₅r¹⁰+α₆r¹²+ . . .

where at least one of the coefficients an is non-zero. In some embodiments, the aspheric surface is described by the addition of odd-powered polynomial terms.

A lens may consist of a single optical element or comprise at least two optical elements. A lens may comprise at least two zones, the zones having different optical corrections (i.e., different optical characteristics) than one another (e.g., the regions have different focal lengths).

Another aspect of the invention is directed to a method of facilitating treatment of spherochromatism in a subject's eye, comprising measuring an amount of spherochromatism in the eye, and selecting an ophthalmic lens to reduce the amount of spherochromatism in the ophthalmic optical system. The method may further comprise applying the lens to the eye.

Still another aspect of the invention is directed to a multizonal ophthalmic lens, comprising at least one optic having at least two zones, the zones having different optical corrections (i.e., different optical characteristics), at least one of the zones having at least one aspheric surface, the at least one zone disposed at least partially in a range 0.7 to 1.0 of the normalized clear aperture of the lens, the zone corrected (i.e., configured) such that, when the lens is applied to an average eye, for a bandwidth between a wavelength of 656 nm and a wavelength of 486 nm, the spherochromatism of the zone is less than 25 waves of the center wavelength of the bandwidth. In some embodiments, the lens is a multifocal lens, and the first and second zones have optical powers that are different than one another.

In some embodiments, the at least one zone is disposed entirely in a range 0.7 to 1.0 of the normalized clear aperture of the lens. In some embodiments, the clear aperture of the lens is 6.0 mm. The lens may be a contact lens, an IOL, a corneal inlay or a corneal onlay or other ophthalmic lens. The zone may have a non-zero amount of spherical aberration for at least one wavelength in the bandwidth.

In some embodiments, the lens is corrected (i.e., configured) such that for a bandwidth between a wavelength of 700 nm and a wavelength of 400 nm, the at least one zone has spherochromatism that is less than 25 waves of the center wavelength of the bandwidth.

In some embodiments, the at least one zone provides a polychromatic modulation that is greater than 10% for a test target having a modulation of 100 line-pairs/mm for light in the bandwidth.

In some embodiments, the sag of the at least one zone is described by the

z_{Schmidt}(r)=z_{s tan dard}(r)+α₁r²+α₂r⁴+α₃r⁶+α₄r⁸+α₅r¹⁰+α₆r¹²+ . . .

equation

where at least one of the coefficients αₙ is non-zero.

In some embodiments, the sag of the zone is described by the addition of at least one odd-powered polynomial term.

Yet another aspect of the invention is directed to a surgical method, comprising providing an ophthalmic lens comprising at least one optic having at least one aspheric surface, the lens corrected (i.e., configured) such that, when the lens is applied to an average eye, for a bandwidth between a wavelength of 656 nm and a wavelength of 486 nm, at locations disposed in a range 0.7 to 1.0 of the normalized clear aperture of the lens, the spherochromatism is less than 25 waves of the center wavelength of the bandwidth. The method also comprising applying the lens to a patient's eye.

In some embodiments, the clear aperture is determined by the region of the lens that is optically corrected (i.e., configured for vision). In other embodiments, the clear aperture is determined by a feature of the eye. In such embodiments, the feature may be the iris.

In some embodiments, the maximum clear aperture when the lens is applied to the eye is 6 mm. In some embodiments, the lens is an IOL and the step of applying comprises inserting the lens in the patient's eye (e.g., using forceps or an IOL injector). In other embodiments, the lens is a contact lens and the step of applying comprises placing the lens on the cornea.

Another aspect of the invention is directed to an ophthalmic lens comprising at least one optic having at least one aspheric surface, the lens corrected (i.e., configured) such that, when the lens is applied to an average eye, for a bandwidth between a wavelength of 656 nm and a wavelength of 486 nm, at a location disposed in a range 0.7 to 1.0 of the normalized clear aperture of the lens, the spherochromatism is less than 25 waves of the center wavelength of the bandwidth.

As used herein, the term “spherochromatism,” for a given bandwidth, refers to the optical path difference (OPD) (measured at an image plane (i.e., with the retina disposed at a plane of best focus)) that occurs between a first ray of light having a wavelength equal to longest wavelength in the bandwidth and a second ray of light having a wavelength equal to shortest wavelength in the bandwidth, the rays passing through a common point in the clear aperture of the lens.

As used herein, the term “positive spherochromatism” means that the lens generates an OPD of light in a bandwidth such that, the short wavelengths of light focus nearer to the lens than longer wavelengths; and the term “negative spherochromatism” of that the lens generates an OPD of light in a bandwidth such that, the longer wavelengths of light focus nearer to the lens than shorter wavelengths.

The unit “waves,” as used herein to specify an amount of spherochromatism, means a length equal to a multiple of a selected wavelength. Unless specified otherwise, the selected wavelength is the center wavelength of a selected bandwidth. The amount is measured at the image plane.

A prescription defining an “average eye” as the term is used herein, and which is to be used to determine performance of lenses as described herein, is provided in Table 5. It will be recognized that the prescription is an eye model according to the Liou and Brennan eye model of 1997.

TABLE 5

| Radius of | | | |
|---|---|---|---|
| Curv. (mm) | Conic | Thickness (mm) | Medium |
| 7.770000 | −0.180000 | 0.500000 | Cornea |
| 6.400000 | −0.600000 | 3.160000 | Aqueous Humor |
| Infinity | 0.000000 | 0.000000 | Iris |
| 12.400000 | −0.940000 | 1.590000 | Anterior Crystalline |
| Infinity | 0.000000 | 2.430000 | Posterior Crystalline |
| −8.100000 | 0.960000 | 16.238830 | Vitreous Humor |
| −13.400000 | 0.000000 | — | — |

It will be appreciated that design and/or performance determination of contact lenses can be achieved with the lens having zero separation from the corneal surface. It will also be appreciated that performance determinations of an intraocular lens (IOL) to be placed in the posterior capsule bag of the lens shall be achieved with the IOL being disposed halfway between the anterior and posterior crystalline lens surfaces (and the optical power of said anterior and posterior surfaces removed). It will be appreciated that lenses located at various positions in the capsular bag will have substantially the same optical performance; such a performance characteristics is typically desirable since precise placement during surgery is difficult.

The above lens locations (e.g., on the cornea or in the capsular bag) are described by way of example. Any other lens can be located at any suitable location in the eye model. The diameter of the iris in the average eye is not specified by the average eye, but may be separately specified.

As one of ordinary skill in the art would understand, to determine performance of a lens of a particular diopteric power (or to design a lens of a particular diopteric power) the model eye should be adjusted such that a best-focus image is obtained on the retina of the model eye. Although multiple, substantially equivalent techniques may be used to achieve best focus on the retina, for purposes of aspects of this invention, the position of the retina can be adjusted (i.e., by changing the depth of the vitreous humor between the posterior lens surface and the retina) to achieve best focus on the retina.

### BRIEF DESCRIPTION OF THE DRAWINGS

Illustrative, non-limiting embodiments of the present invention will be described by way of example with reference to the accompanying drawings, in which the same reference number is used to designate the same or similar components in different figures, and in which:

FIG. 1A is a plan view of an example of an ophthalmic lens according to aspects of the present invention;

FIG. 1B is a cross sectional side view of the ophthalmic lens shown in FIG. 1A;

FIGS. 2A and 2B are schematic illustrations of a lens having spherical aberration with light of two wavelengths passing therethrough, showing that light passing through the 0.0-0.7 portion of a normalized portion of a pupil of the lens has less of an impact on the lateral deviation of color than light passing through a portion of the lens disposed at the greater than 0.7 of the normalized clear aperture;

FIGS. 3 and 4 are OPD and MTF plots, respectively, corresponding to a first example of an embodiment of a lens according to aspects of the invention, showing performance when the lens is applied to an average eye;

FIGS. 5 and 6 are OPD and MTF plots, respectively, corresponding to a second example of an embodiment of a lens according to aspects of the invention, showing performance when the lens is applied to an average eye;

FIGS. 7 and 8 are OPD and MTF plots, respectively, corresponding to a third example of an embodiment of a lens according to aspects of the invention, showing performance when the lens is applied to an average eye;

FIGS. 9 and 10 are OPD and MTF plots corresponding to a fourth example of an embodiment of a lens according to aspects of the invention, showing performance when the lens is applied to an average eye; and

FIG. 11 is a plan view of an example of a multizonal lens according to aspects of the present invention.

### DETAILED DESCRIPTION

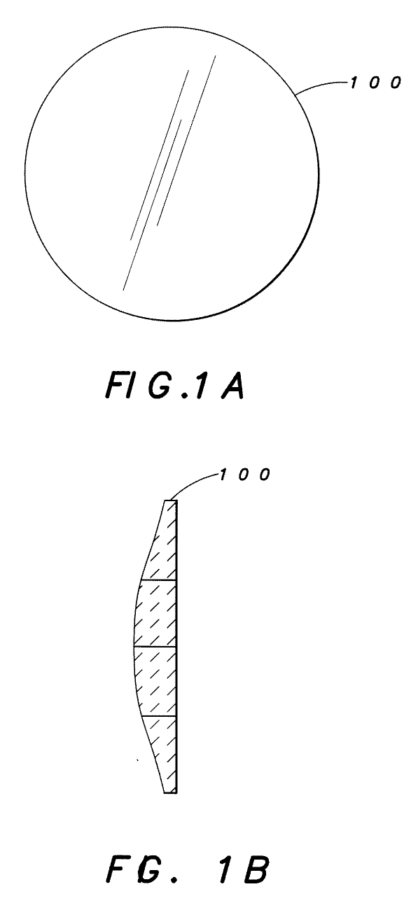

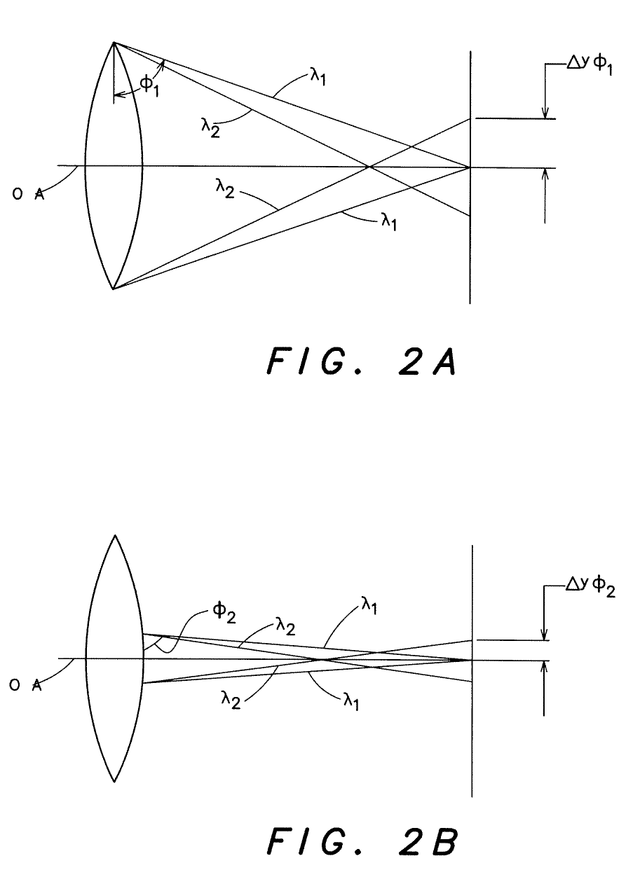

FIG. 1A is a plan view of an example of an ophthalmic lens 100 according to aspects of the present invention. It will be appreciated that non-optical components of the lens may be added in some embodiments (e.g., in intraocular lenses, haptics may be added). FIG. 1B is a cross sectional side view of ophthalmic lens 100 according to aspects of the present invention.

As discussed above, according to some aspects of the invention, an ophthalmic lens comprises at least one lens having at least one aspheric surface. The lens is corrected (i.e., configured) such that, when applied to an average eye, for a bandwidth of light between a wavelength of 656 nm and a wavelength of 486 nm, at locations disposed in a range 0.7 to 1.0 of the normalized clear aperture of the lens, the spherochromatism is less than 25 waves of the center wavelength of the bandwidth. It will be appreciated that, although in some embodiments correction (i.e., control) is achieved over a range of the clear aperture, correction at one or more selected locations in the clear aperture may provide advantages.

It will be appreciated that, in lenses according to aspects of the invention, for rays of a relatively long wavelength (e.g., at least 656 nm) and rays of a relatively short wavelength (e.g., at most 486 nm) emanating from a common point in the clear aperture, the optical path difference is equal to less than 25 waves. In some embodiments, it may be desirable that the difference is less than 20 waves; in other embodiments, it may be desirable that the difference is less than 10 waves; and in still other embodiments, it may be desirable that the difference is less than 5.0 waves. In some embodiments, the difference is less than 1.0 wave. Each reduction in spherochromatism represents an improvement in lens performance (e.g., a decrease in haloing seen by a wearer).

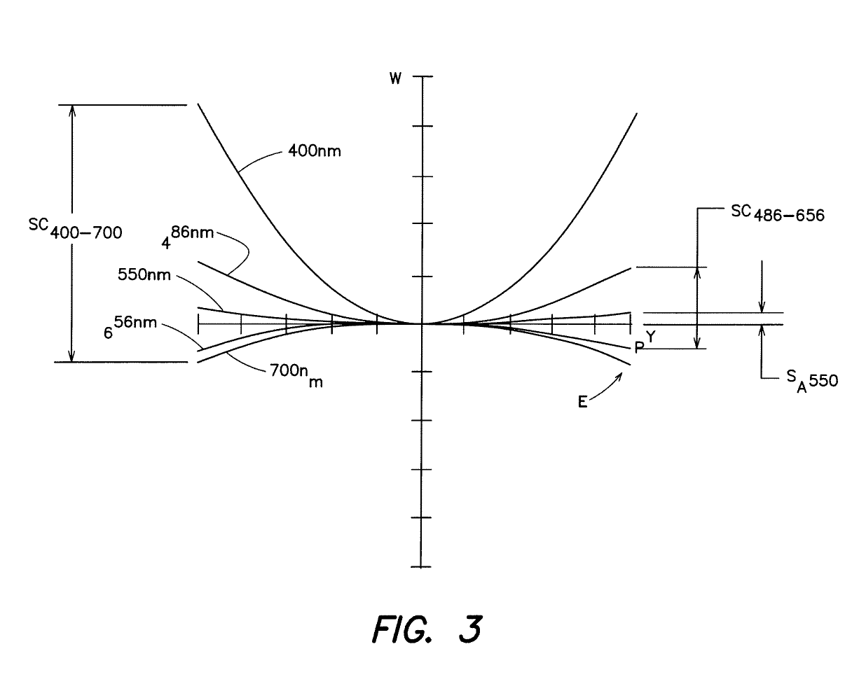

The deviation of rays of the longest and shortest wavelength are measured for rays emanating from a common point disposed in a range of 0.7 of the normalized clear aperture of the lens (i.e., approximately a radial distance of 2.1 mm (measured from the lens vertex) in a standard lens having a 6.0 mm clear aperture diameter) to the edge of the clear aperture (i.e., 1.0 of the normalized clear aperture, a radial distance of 3.0 mm in a standard lens having a 6.0 mm clear aperture diameter). It is first to be appreciated that, if light in a range of 0.70-1.0 of the normalized clear aperture is suitably corrected (i.e., controlled), light at points radially inward of 0.7 of the normalized clear aperture (i.e., light that is more paraxial) will also typically be suitably corrected. However, in any given embodiment, it will also be appreciated that light in the range 0.0-0.70 may have more or less spherochromatism than the light in the range 0.70-1.0. As illustrated in FIGS. 2A and 2B, due to the relatively small angle of convergence Ø₂ of light in the range 0.0-0.7, the overall impact on the lateral deviation of color (Δy_{Ø2}) of the image is relatively small compared to the impact on lateral deviation of color (Δy_{Ø1}) for light in the range 0.7 of the clear aperture and greater, and having an angle of convergence Ø₁. As one of ordinary skill in the art will understand, for a lens having a circular clear aperture, the light transmitted within 0.0-0.7 of the normalized clear aperture, will contain 50% of the energy transmitted through the clear aperture and is a common demarcation for specifying optical performance. Lenses according to aspects of the present invention are not limited to those having a circular clear aperture.

Although the example above includes numbers for an ophthalmic lens having a clear aperture diameter of 6.0 mm, ophthalmic lenses according to aspects of the present invention can have any suitable clear aperture diameter. For example, diameters may be in the range of 2-6 mm (e.g., 2 mm, 3 mm, 4 mm, 5 mm or 6 mm). It will be appreciated that it is generally easier to correct (i.e., control) aberrations for a lens that has a smaller diameter.

In some embodiments, the boundary of the clear aperture of a lens is determined by edges of the region of a lens that are suitable for vision (i.e., the clear aperture is the image forming portion of the lens). In other embodiments, the boundary of the clear aperture is determined by a natural or implanted feature of the eye (e.g., an iris or implanted ring) in which the lens is placed (e.g., the maximum diameter of a wearer's iris can be 6 mm). It will also be appreciated that, if a feature of the eye will determine the clear aperture, the diameter of the clear aperture is at least partially determined by the location of the lens in the eye relative to the location of the feature (e.g., a contact lens adapted be located on a cornea will have different clear aperture than the clear aperture of an IOL adapted to be located in a posterior chamber of the eye).

In some embodiments, the ophthalmic lens is adapted to provide suitable spherochromatism characteristics as set forth above after the lens is applied to the eye, the maximum clear aperture being 6 mm. It will be appreciated that, as described above, the clear aperture of the lens, when applied to the eye, may be determined by the size of the imaging forming portion of the lens or a feature of the eye. In other embodiments, the ophthalmic lens is adapted to provide suitable spherochromatism characteristics as set forth above after the lens is applied to the eye, the lens having a maximum clear aperture of 5 mm; in yet other embodiments, the ophthalmic lens is adapted to provide suitable spherochromatism characteristics as set forth above after the lens is applied to the eye, the lens having a maximum clear aperture of 4 mm.

It is also to be appreciated that, correction (i.e., control) of spherochromatism does not require full correction (i.e., substantially zero waves) of spherical aberration of a lens. In some embodiments, it is desirable to correct spherical aberration and spherochromatism; however, given the limited number of degrees of freedom in an ophthalmic lens it may not be possible to fully correct, both, spherical aberration for all wavelengths within a given bandwidth, and spherochromatism.

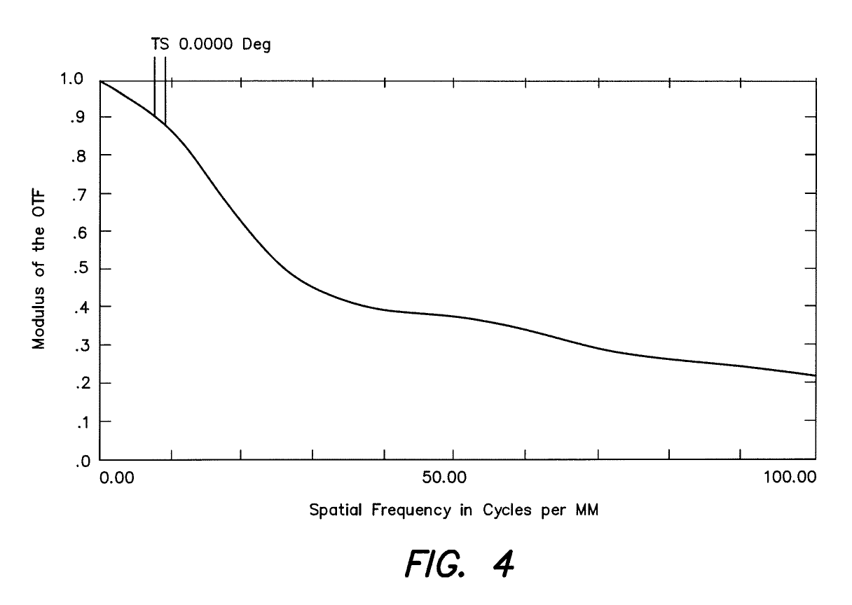

In other embodiments, it is desirable to have a non-zero amount of spherical aberration to provide a lens with depth of field, and thereby ameliorate a wearer's presbyopia. In some embodiments, at one or more wavelengths of the visible bandwidth, the lens has spherical aberration of at least 1 wave. In other embodiments, the lens has spherical aberration at one or more wavelengths of at least 2 waves; and in still other embodiments at least 5 waves at one or more wavelengths. One such embodiment is illustrated in FIG. 3. In the illustrated embodiment, spherical aberration is demonstrated by the parabolic shape of the OPD plot for a single wavelength light. For the plots illustrated in FIG. 3, the magnitude of spherical aberration of 486 nm light is 4 wavelengths at the edge (E) of the clear aperture; and the magnitude of the spherical aberration of the 700 nm light is 3 wavelengths at the edge of the clear aperture. It will also be appreciated that, by not fully correcting spherical aberration, the degrees of freedom available to a designer to correct (i.e., control) other aberrations is increased.

Rays having a wavelength between 656 nm and 486 nm represent a substantial portion of the visible spectrum (particularly in older subjects) including those to which the human eye is most sensitive; and said wavelengths are commonly used design wavelengths for ophthalmic lenses. However, other bandwidths may be used. For example, wavelengths between 400 nm and 700 nm may be used, which include approximately the entire visible spectrum for a healthy human eye. Generally, specifying an OPD of a given magnitude for a selected bandwidth which has a longer wavelength at the long wavelength end of the bandwidth or a shorter wavelength at the short wavelength end of the bandwidth will put a greater constraint on performance, such that a greater percentage of the visible light energy will arrive in a given image spot.

In some instances, it is appropriate to further determine suitability of an ophthalmic lens by observing the polychromatic modulation transfer function (MTF) of the lens. For example, in lenses according to some aspects of the present invention, light across the selected bandwidth of the lens, the modulation is greater than 10% for a test target of 100 line-pairs/mm. It will be appreciated that modulation transfer generally increases with decreasing spatial frequency so that the modulation transfer will be greater than 10% for targets having a modulation of less than 100 line-pairs/mm.

In some embodiments of the present invention, to achieve suitable performance characteristics, a lens includes at least one aspheric surface whose sag is defined by the following equation

$${z}_{standard} ( r ) = \frac{{cr}^{2}}{1 + \sqrt{1 - ( 1 + k ) {c}^{2} {r}^{2}}}$$

where c is the curvature of said surface k the conic constant and r a radial coordinate. If k=0, then the surface would be spherical. The surface is described by a sag as follows:

z(r)=z_{s tan dard}(r)+α₁r²+α₂r⁴+α₃r⁶+α₄r⁸+α₅r¹⁰+α₆r¹²+ . . .

It will be appreciated that the equation includes a conic and even-powered polynomial terms. According to aspects of the present invention at least one of the αₙ terms is non-zero. It will be understood that it is typically desirable that the number of αₙ terms selected to be non-zero be the minimum necessary to achieve a selected performance.

It will also be appreciated that, in some embodiments, to achieve a desired result, embodiments of the present invention include even-powered polynomial components (as presented above). Such embodiments are capable of providing performance beyond lenses having surfaces with only a standard conic asphere (z_{standard}). In some embodiments, the lenses include surfaces having only even-powered aspheric terms. It is further to be appreciated that although even-powered polynomial terms may be all that is necessary to achieve correction (i.e., suitable control) for a lens that is to have its optical axis aligned with the visual axis of a patient, for embodiments that are to be used in a non-aligned arrangement, odd-powered polynomial terms may be added. For example, odd-powered aspheric terms may be appropriately used with contact lenses embodiments, where decentration is likely.

Examples of lens prescriptions providing suitable performance characteristics according to aspects of the present invention are provided below. The embodiments were designed using Zemax version Jan. 22, 2007. Zemax design software is available from Zemax Development Corporation of Bellevue, Wash.

### EXAMPLE 1

Table 1 is a prescription for a first example of an embodiment of a lens according to aspects of the present invention. Table 1 illustrates an example of a single-element, intraocular lens (IOL) made of an example hydrophilic acrylic material having an index of refraction equal to approximately 1.46 for the d-wavelength of 0.589 micrometers; and as a function of wavelength (λ), n equals 1.38529196+(1.12901134E-002/λ)+(2.29091649E-004/(λ)^{3.5}), where wavelength is given in microns.

TABLE 1

| | Radius | Conic | Thickness | | | | |
|---|---|---|---|---|---|---|---|
| Surface | R (mm) | Constant k | (mm) | α₁ (mm) | α₂ (mm) | α₃ (mm) | α₄ (mm) |
| 1 | 5.125254 | −1.701344 | 0.654523 | 0.012439 | −2.11E−03 | −2.02E−04 | −6.85E−06 |
| 2 | 1.20E+04 | 2.08E+07 | — | — | — | — | — |

The lens described in Table 1 has two aspheric surfaces. The first surface includes only even-powered aspheric terms (in addition to a conic term).

An OPD plot illustrating the on-axis spherochromatism performance of the lens of Table 1 in a capsular bag of an average eye is shown in FIG. 3 (where the vertical axis maximum scale is ±20 waves). As is apparent from the OPD plot, for a bandwidth between a wavelength of 700 nm and a wavelength of 400 nm, at locations disposed in a range 0.7 to 1.0 of the normalized clear aperture of the lens, the spherochromatism SC₄₀₀₋₇₀₀ is less than about 20 waves.

Also apparent from the OPD plot, for a bandwidth between a wavelength of 656 nm and a wavelength of 486 nm, at locations disposed in a range 0.7 to 1.0 of the normalized clear aperture of the lens, the spherochromatism SC₄₈₆₋₆₅₆ is less than about 7 waves.

The spherical aberration SA₅₅₀ of the lens is about 1 wave for the center wavelength. The spherical aberration of the lens is about 4 waves for 486 nm light. The spherical aberration of the lens is about 3 waves for 656 nm light. The diameter of the clear aperture of the lens is 6 mm.

A polychromatic MTF plot (for light in the bandwidth 486 nm to 656 nm) for the lens specified in Table 1 applied to an average eye is shown in FIG. 4. The plot illustrates that modulation is greater than 20% for an object having 100 lp/mm or less.

### EXAMPLE 2

Table 2 is a prescription for another example of an embodiment of a lens according to aspects of the present invention. Table 2 illustrates another example of a single-element, IOL made of the same hydrophylic material as Example 1 and corrected (i.e., configured) according to aspects of the present invention.

TABLE 2

| | Radius | Conic | Thickness | | | | |
|---|---|---|---|---|---|---|---|
| Surface | R (mm) | Constant k | (mm) | α₁ (mm) | α₂ (mm) | α₃ (mm) | α₄ (mm) |
| 1 | 7.505389 | 3.370367 | 0.524952 | 0.090794 | 1.035E−03 | −8.99E−05 | 1.45E−05 |
| 2 | 6.423080 | 5.106641 | | | | | |

The lens described in Table 2 has two aspheric surfaces. The first surface includes only even-powered aspheric terms (in addition to a conic term).

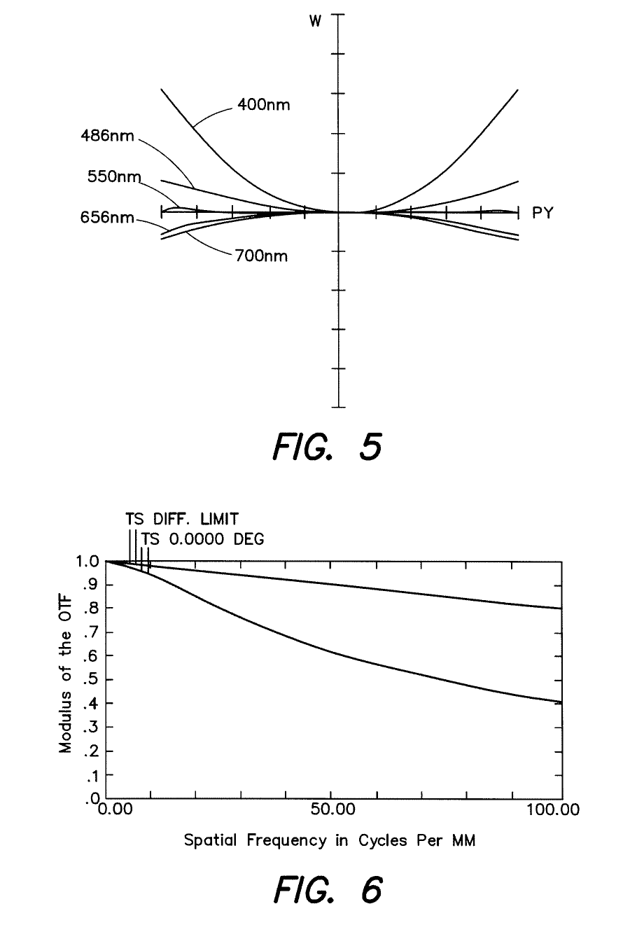

An OPD plot illustrating the on-axis spherochromatism performance of the lens of Table 2 applied to an average eye is shown in FIG. 5 (where the vertical axis maximum scale is ±20 waves). As is apparent from the OPD plot, for a bandwidth between a wavelength of 700 nm and a wavelength of 400 nm, at locations disposed in a range 0.7 to 1.0 of the normalized clear aperture of the lens, the spherochromatism is about 16 waves.

Also apparent from the OPD plot, for a bandwidth between a wavelength of 656 nm or greater and a wavelength of 486 nm or less, at locations disposed in a range 0.7 to 1.0 of the normalized clear aperture of the lens, the spherochromatism is less than about 6 waves at the center wavelength of the bandwidth (571 nm).

The spherical aberration of the lens is less than 1 wave for the center wavelength. The spherical aberration of the lens is about 4 waves for 486 nm light. The spherical aberration of the lens is about 3 waves for 656 nm light. The diameter of the clear aperture of the lens is 6 mm.

A polychromatic MTF plot (for light in the bandwidth 400 nm to 700 nm) of the lens specified in Table 2 applied to an average eye (i.e., disposed in a capsular bag) is shown in FIG. 6. The plot illustrates that modulation is greater than 40% for an object having 100 lp/mm or less.

### EXAMPLE 3

Table 3 is a prescription for an example of a single-element, contact lens made of a Silicon Hydrogel material having an index of refraction (n) equal to approximately 1.41 for the d-wavelength of 0.589 micrometers; and as a function of wavelength (λ), n equals 1.43892094+(1.10429710E-002/λ)+(2.49170954E-004/(λ)^{3.5}), where wavelength is given in microns.

TABLE 3

| | Radius | Conic | Thickness | | | | |
|---|---|---|---|---|---|---|---|
| Surface | R (mm) | Constant k | (mm) | α₁ (mm) | α₂ (mm) | α₃ (mm) | α₄ (mm) |
| 1 | 8.202788 | −1.799671 | 0.150000 | −4.98E−04 | 2.503E−04 | 1.12E−06 | −2.70E−08 |
| 2 | 7.800000 | −0.250000 | — | — | — | — | — |

The lens described in Table 3 has two aspheric surfaces. The first surface includes only even-powered aspheric terms.

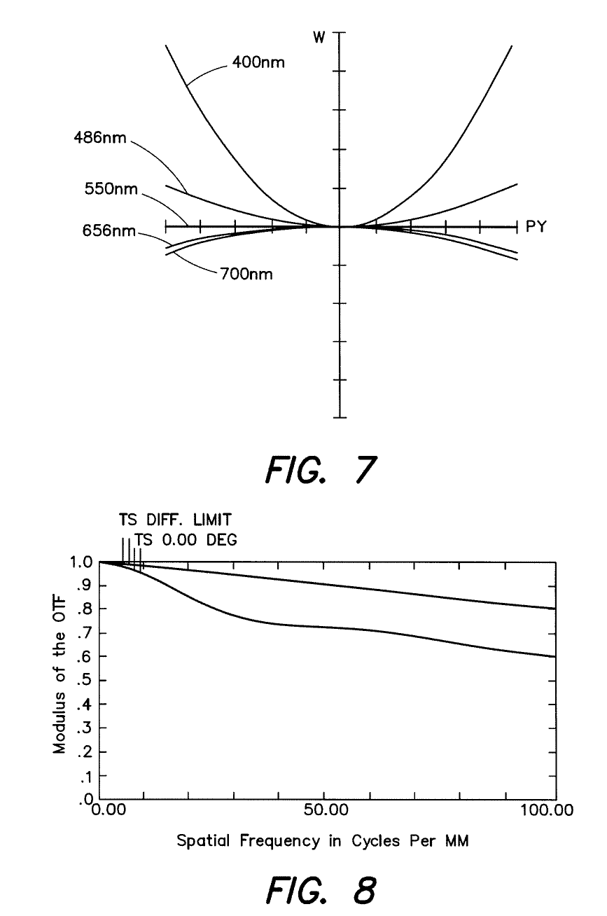

An OPD plot illustrating the on-axis spherochromatism performance of the lens of Table 3 applied to an average eye is shown in FIG. 7 (where the vertical axis maximum scale is ±20 waves). As is apparent from the OPD plot, for a bandwidth between a wavelength of 700 nm and a wavelength of 400 nm, at locations disposed in a range 0.7 to 1.0 of the normalized clear aperture of the lens, the spherochromatism is about 21 waves.

Also apparent from the OPD plot, for a bandwidth between a wavelength of 656 nm or greater and a wavelength of 486 nm or less, at locations disposed in a range 0.7 to 1.0 of the normalized clear aperture of the lens, the spherochromatism is less than 6 waves.

The spherical aberration of the lens is less than 1 wave for the center wavelength. The spherical aberration of the lens is about 4 waves for 486 nm light. The spherical aberration of the lens is about 2 waves for 656 nm light. The diameter of the clear aperture of the lens is 6 mm.

A polychromatic MTF plot (for light in the bandwidth 400 nm to 700 nm) of the lens specified in Table 3 applied to an average eye is shown in FIG. 8. The plot illustrates that modulation is greater than 40% for an object having 100 lp/mm or less.

### EXAMPLE 4

Table 4 is a prescription for an example of a dual-element lens made of an example silicone material corrected (i.e., configured) according to aspects of the present invention.

TABLE 4

| | Radius | Conic | Thickness | | | | |
|---|---|---|---|---|---|---|---|
| Surface | R (mm) | Constant k | (mm) | α₁ (mm) | α₂ (mm) | α₃ (mm) | α₄ (mm) |
| 1 | 3.782992 | −0.062781 | 1.420777 | 2.86E−05 | 1.85E−03 | −1.67E−07 | −1.79E−04 |
| 2 | −6.877061 | 0.618306 | — | — | — | — | — |
| 3 | −4.641906 | −2.376222 | 0.200 | — | — | — | — |
| 4 | −115.941 | 1061.732833 | — | 0.010661 | −7.35E−04 | −1.56E−03 | 1.00E−03 |

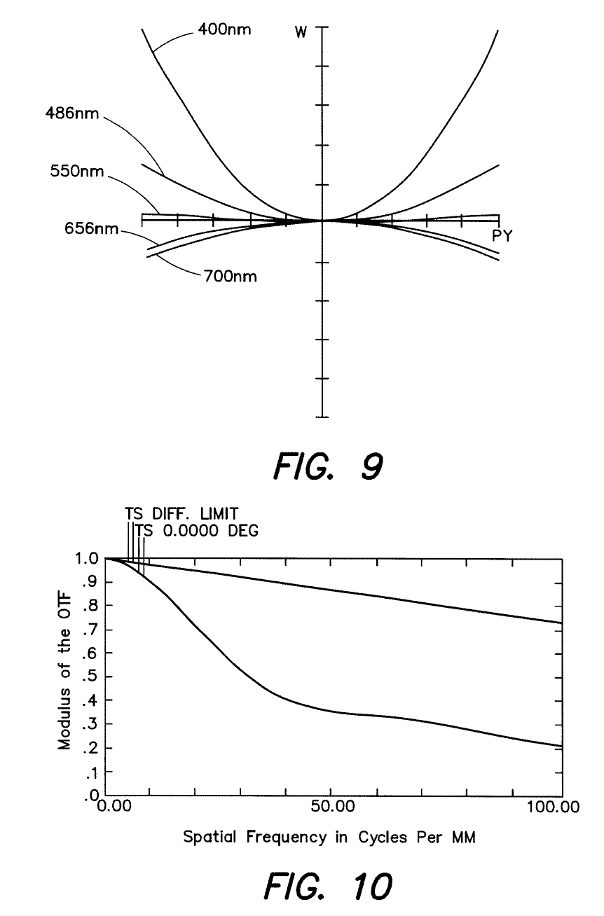

An OPD fan illustrating the on-axis spherochromatism performance of the lens of Table 4 applied to an average eye is shown in FIG. 9 (where the vertical axis maximum scale is ±10 waves). As is apparent from the OPD plot, for a bandwidth between a wavelength of 700 nm and a wavelength of 400 nm, at locations disposed in a range 0.7 to 1.0 of the normalized clear aperture of the lens, the spherochromatism is about 11 waves.

Also apparent from the OPD plot, for a bandwidth between a wavelength of 656 nm and a wavelength of 486 nm, at locations disposed in a range 0.7 to 1.0 of the normalized clear aperture of the lens, the spherochromatism is about 4 waves.

The spherical aberration of the lens is about 0.5 at the center wavelength. The spherical aberration of the lens is about 3 waves for 486 nm light. The spherical aberration of the lens is about 1 wave for 656 nm light. The diameter of the lens is 6 mm.

A polychromatic MTF plot (for light in the bandwidth 486 nm to 656 nm) of the lens specified in Table 4 applied to an average eye is shown in FIG. 10. The plot illustrates that modulation is greater than 20% for an object having 100 lp/mm or less.

According to another aspect of the invention, a patient's spherochromatism is measured, for example, using an aberrometer (e.g., a Hartmann Shack aberrometer) capable of measuring aberrations at at least two wavelengths. And an ophthalmic lens having a desired amount of spherochromatism correction (i.e., control) is manufactured. The lens may have positive or negative spherochromatism. It will be appreciated that such correction may be provided with or without correction of other aberrations. The ophthalmic lens may be any suitable lens according to the disclosure herein. The lens may be applied to (e.g., deposited on, implanted in or attached to) the patient's eye. As one of ordinary skill in the art would understand, a suitable technique will be selected according to the type of lens.

Another aspect of the invention is directed to a multifocal lens having spherochromatic correction (i.e., control). It will be appreciated that, although the above designs indicate optics having only a single region having a single nominal focal length, aspects of the present invention may be applied to a lens having two or more zones having different optical corrections (i.e., different optical characteristics). In some embodiments the lens is multifocal and the two or more zones have different nominal focal lengths (i.e., the lens is a multifocal lens). For example, to achieve such a lens, a design as set forth above may be formed into an annulus by eliminating a central portion of the lens and replacing the central portion with one or more portions each having an appropriate add or subtract power to achieve multifocal vision. The outer portions of the lens may also (or instead) be replaced with one or more zones, which may or may not be corrected for spherochromatism. Although the above discussion is with reference to a lens having annular zones, any suitable arrangement of zones may be used (e.g., non-circularly symmetric zones).

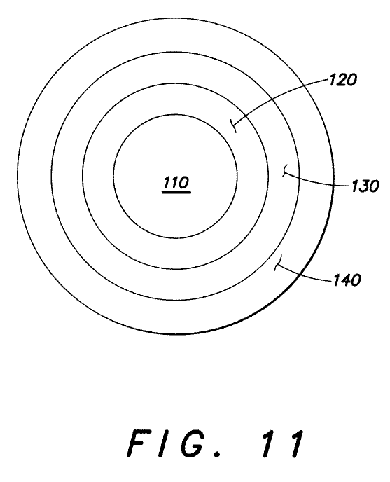

FIG. 11 is an illustration of one example of a lens according to aspects of the present invention having zones 110, 120, 130 and 140. The zones have different focal lengths. It will be appreciated that the presence of two or more zones in a lens can be recognized by a presence of commensurate number of local maxima in a plot of optical response (e.g., contrast, strehl ratio, resolution) as a function of vergence.

As stated above, multifocal lenses are particularly susceptible to spherochromatism (i.e., color haloing) because, for example, all of the light at a particular focal distance (or a substantial portion of the light at the focal distance) may come from a location that is apart from the optical axis of the lens (e.g., 0.7 to 1.0 of the clear aperture) meaning that spherochromatism is particularly large for all of the light at the focal distance.

According to aspects of the invention, at least one of the zones has at least one aspheric surface, and is corrected (i.e., configured) such that, when the lens is applied to an average eye, for a bandwidth between a wavelength of 656 nm and a wavelength of 486 nm, the spherochromatism of the zone is less than 25 waves of the center wavelength of the bandwidth. In some embodiments, the zone is configured such that for a bandwidth between a wavelength of 700 nm and a wavelength of 400 nm, the spherochromatism of the zone is less than 20 waves of the center wavelength of the bandwidth. The spherochromatism may be less than 10 waves; and in some embodiments, less than 5 waves, in other embodiments less than one wave.

Typically the corrected zone (i.e., the zone having controlled spherochromatism aberration) will be apart from the optical axis (i.e., the zone is non-axial). In some embodiments, at least one of the zone is disposed at least partially in a range 0.7 to 1.0 of the normalized clear aperture of the lens. In some embodiments, the zone that is corrected (i.e., the zone having controlled spherochromatism aberration) is located entirely within 0.7 to 1.0 of the normalized clear aperture.

Although the lens is illustrated with four zones, lenses according to aspects of the present invention have two or three or more zones. As described above, the zone may have a non-zero amount of spherical aberration for at least one wavelength in the bandwidth. The magnitude of the spherical aberration may be at least one wave, at least two waves or at least five waves. The sag of the surface may be as described by the sag equation above. The sag may include only positive-powered polynomial terms or may include one or more negative-powered polynomial terms.

In some instances, it is appropriate to further determine suitability of an ophthalmic lens by observing the polychromatic modulation transfer function (MTF) of a zone. For example, according to some aspects of the present invention, for a corrected zone, light across the selected bandwidth of the lens has a modulation that is greater than 10% for a test target of 100 line-pairs/mm or less.

Lenses according to aspects of the present invention may be applied to an eye using a suitable technique. For example, a contact lens may be placed on the cornea; and an IOL may be inserted into the eye through an incision in the sclera.

Having thus described the inventive concepts and a number of exemplary embodiments, it will be apparent to those skilled in the art that the invention may be implemented in various ways, and that modifications and improvements will readily occur to such persons. Thus, the embodiments are not intended to be limiting and presented by way of example only. The invention is limited only as required by the following claims and equivalents thereto.

What is claimed is:

1. An ophthalmic lens, comprising:
at least one optic having at least one aspheric surface, the lens configured such that, when the lens is applied to an average eye, for a bandwidth between a wavelength of 656 nm and a wavelength of 486 nm, at locations disposed in a range 0.7 to 1.0 of the normalized clear aperture of the lens, the spherochromatism is less than 25 waves of the center wavelength of the bandwidth.

2. The lens of claim 1, wherein the spherochromatism is less than 20 waves.

3. The lens of claim 1, wherein the spherochromatism is less than 10 waves.

4. The lens of claim 1, wherein the spherochromatism is less than 1 wave.

5. The lens of claim 1, wherein the spherochromatism is less than 0.5 waves.

6. The lens of claim 1, wherein the clear aperture of the lens is 6.0 mm.

7. The lens of claim 1, wherein the clear aperture of the lens is 5.0 mm.

8. The lens of claim 1, wherein the clear aperture of the lens is 4.0 mm.

9. The lens of claim 1, wherein the clear aperture of the lens is 3.0 mm.

10. The lens of claim 1, wherein the clear aperture of the lens is 2.0 mm.

11. The lens of claim 1, wherein the lens is a contact lens.

12. The lens of claim 1, wherein the lens is an IOL.

13. The lens of claim 1, wherein the lens is a corneal inlay or a corneal onlay.

14. The lens of claim 1, wherein the lens has a non-zero amount of spherical aberration for at least one wavelength in the bandwidth.

15. The lens of claim 14, wherein the amount of spherical aberration is at least one wave.

16. The lens of claim 14, wherein the amount of spherical aberration is at least 2 waves.

17. The lens of claim 14, wherein the amount of spherical aberration is at least 5 waves.

18. The lens of claim 1, wherein the lens is configured such that for a bandwidth between a wavelength of 700 nm and a wavelength of 400 nm, at locations disposed in a range 0.7 to 1.0 of the normalized clear aperture of the lens, the spherochromatism being less than 25 waves of the center wavelength of the bandwidth.

19. The lens of claim 1, wherein the lens provides a polychromatic modulation that is greater than 10% for a test target having a modulation of 100 line-pairs/mm for light in the bandwidth.

20. The lens of claim 1, wherein the sag of the aspheric surface is described by
z_{Schmidt}(r)=z_{s tan dard}(r)+α₁r²+α₂r⁴+α₃r⁶+α₄r⁸+α₅r¹⁰+α₆r¹²+ . . .
where at least one of the coefficients αₙ is non-zero.

21. The lens of claim 20, wherein the lens is described by the addition of at least one odd-powered polynomial term.

22. The lens of claim 1, wherein the lens consists of a single optical element.

23. The lens of claim 1, wherein the lens comprises at least two optical elements.

24. The lens of claim 1, wherein the lens comprises at least two regions, the regions having different nominal focal lengths than one another.

25. A method of facilitating treatment of spherochromatism in a subject's eye, comprising:
measuring an amount of spherochromatism of the eye; and
selecting an ophthalmic lens to reduce the amount of spherochromatism.

26. A multizonal ophthalmic lens, comprising:
at least one optic having at least two zones, at least one of the zones having at least one aspheric surface, the at least one zone disposed at least partially in a range 0.7 to 1.0 of the normalized clear aperture of the lens, the at least one zone configured such that, when the lens is applied to an average eye, for a bandwidth between a wavelength of 656 nm and a wavelength of 486 nm, the spherochromatism of the zone is less than 25 waves of the center wavelength of the bandwidth.

27. The lens of claim 26, wherein the at least one zone is disposed entirely in a range 0.7 to 1.0 of the normalized clear aperture of the lens.

28. The lens of claim 26, wherein the spherochromatism is less than 10 waves.

29. The lens of claim 26, wherein the spherochromatism is less than 5 waves.

30. The lens of claim 26, wherein the clear aperture of the lens is 6.0 mm.

31. The lens of claim 26, wherein the lens is a contact lens.

32. The lens of claim 26, wherein the lens is an IOL.

33. The lens of claim 26, wherein the lens is a corneal inlay or a corneal onlay.

34. The lens of claim 26, wherein the at least one zone has a non-zero amount of spherical aberration for at least one wavelength in the bandwidth.

35. The lens of claim 34, wherein the amount of spherical aberration is at least one wave.

36. The lens of claim 34, wherein the amount of spherical aberration is at least 2 waves.

37. The lens of claim 34, wherein the amount of spherical aberration is at least 5 waves.

38. The lens of claim 26, wherein the lens is configured such that for a bandwidth between a wavelength of 700 nm and a wavelength of 400 nm, the at least one zone has spherochromatism that is less than 25 waves of the center wavelength of the bandwidth.

39. The lens of claim 26, the at least one zone provides a polychromatic modulation that is greater than 10% for a test target having a modulation of 100 line-pairs/mm for light in the bandwidth.

40. The lens of claim 26, wherein the sag of the at least one zone is described by
z_{Schmidt}(r)=z_{s tan dard}(r)+α₁r²+α₂r⁴+α₃r⁶+α₄r⁸+α₅r¹⁰+α₆r¹²+ . . .
where at least one of the coefficients αₙ is non-zero.

41. The lens of claim 40, wherein the sag of the zone is described by the addition of at least one odd-powered polynomial term.

42. The lens of claim 41, wherein the multizonal lens is a multifocal lens and the first and the second zones have different optical powers than one another.

43. A surgical method, comprising:
providing an ophthalmic lens, comprising at least one optic having at least one aspheric surface, the lens configured such that, when the lens is applied to an average eye, for a bandwidth between a wavelength of 656 nm and a wavelength of 486 nm, at locations disposed in a range 0.7 to 1.0 of the normalized clear aperture of the lens, the spherochromatism being less than 25 waves of the center wavelength of the bandwidth; and
applying the lens to a patient's eye.

44. The method of claim 43, wherein the clear aperture is determined by a feature of the eye.

45. The method of claim 43, wherein the feature is an iris of the eye.

46. The method of claim 43, wherein the maximum clear aperture when applied to the eye is 6 mm.

47. The method of claim 43, wherein the maximum clear aperture when applied to the eye is 5 mm.

48. The method of claim 43, wherein the maximum clear aperture when applied to the eye is 4 mm.

49. The method of claim 43, wherein the lens is an IOL and the step of applying comprises inserting the lens in the patient's eye.

50. An ophthalmic lens, comprising:
at least one optic having at least one aspheric surface, the lens configured such that, when the lens is applied to an average eye, for a bandwidth between a wavelength of 656 nm and a wavelength of 486 nm, at a location disposed in a range 0.7 to 1.0 of the normalized clear aperture of the lens, the spherochromatism is less than 25 waves of the center wavelength of the bandwidth.
